(19) United States
(12) Patent Application Publication
(10) Pub. No.: US 2006/0069367 A1
(43) Pub. Date: Mar. 30, 2006

(54) Absorbent core having two or more types of superabsorbent

(76) Inventors: Andrew Waksmundzki, Jackson, NJ (US); John Litvay, Downingtown, PA (US); Frank Glaug, Chester Springs, PA (US)

Correspondence Address: Christopher C. Campbell, Esq.;Hunton & Williams, Suite 1200, 1900 K Street, Washington, DC, 20006-1109, US

(21) Appl. No.: 10/951,888

(22) Filed: Sep. 29, 2004

(51) Int. Cl. A61F 13/15

(52) U.S. Cl. 604/378; 604/368

(57) ABSTRACT

An absorbent core for use in an absorbent article, including a central fibrous layer having synthetic fibers, and two or more types of superabsorbent material (SAP). The SAP is contained primarily in upper and/or lower layers of the absorbent core. The upper and/or lower layer may be a substantially continuous layer of SAP, or may include a free-form of SAP. The upper and/or lower layers may have a blend of two or more types of SAP, or may have only a virgin, unblended SAP. Each of the types of SAP preferably has a different absorbent property (such as high AUL, or fast absorption rate), so that the resultant absorbent core benefits from having multiple absorbent properties provided by the SAP's. An absorbent article including the absorbent core, and a method of providing an absorbent article including the absorbent core are also described.

### BACKGROUND OF THE INVENTION

1. Field of the Invention

The present invention relates generally to an absorbent core for an absorbent article, and more particularly to an absorbent core having synthetic fiber, and two or more types of superabsorbent polymer. The two or more types of superabsorbent polymer have different absorbent properties which are beneficial to the overall absorbent performance of the absorbent core. The two or more types of superabsorbent polymer may be provided in discrete upper and/or lower layers in the absorbent core. Such absorbent cores provide increased absorbency, faster absorbency, and reduced rewet, and allow for reduced amounts of fiber and reduced shifting of SAP particulates within the absorbent core.

2. Description of Related Art

Disposable absorbent garments such as infant diapers or training pants, adult incontinence products and other such products typically were constructed with a moisture-impervious outer backing sheet, a moisture-pervious body-contacting inner liner sheet, and a moisture-absorbent core sandwiched between the liner and backing sheets. Much effort has been expended to find cost-effective materials for absorbent cores that display favorable liquid absorbency and retention. Superabsorbent materials in the form of granules, beads, fibers, bits of film, globules, etc., have been favored for such purposes. Such superabsorbent materials generally are polymeric gelling materials that are capable of absorbing and retaining large quantities of liquid, such as water and body wastes, relative to their own weight, even under moderate pressure.

The superabsorbent material generally is a water-insoluble but water-swellable polymeric substance capable of absorbing water in an amount which is at least ten times the weight of the substance in its dry form. A superabsorbent material comprising a polymer is hereinafter referred to as a superabsorbent polymer or “SAP”. In one type of SAP, the particles or fibers may be described chemically as having a back bone of natural or synthetic polymers with hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Such modified polymers may also be cross-linked to reduce their water-solubility.

The ability of a superabsorbent material to absorb liquid typically is dependent upon the form, position, and/or manner in which particles of the superabsorbent are incorporated into the absorbent core. Whenever a particle of the superabsorbent material and absorbent core is wetted, it swells and forms a gel. Gel formation can block liquid transmission into the interior of the absorbent core, a phenomenon called “gel blocking.” Gel blocking prevents liquid from rapidly diffusing or wicking past the “blocking” particles (e.g., those particles that have swelled and touched an adjacent swelled particle), causing portions of a partially hydrated core to become inaccessible to multiple doses of urine. Further absorption of liquid by the absorbent core must then take place via a diffusion process. This is typically much slower than the rate at which liquid is applied to the core. Gel blocking often leads to leakage from the absorbent article well before all of the absorbent material in the core is fully saturated.

Despite the incidence of gel blocking, superabsorbent materials are commonly incorporated into absorbent cores because they absorb and retain large quantities of liquid, even under load. However, in order for superabsorbent materials to function, the liquid being absorbed in the absorbent structure must be transported to unsaturated superabsorbent material. In other words, the superabsorbent material must be placed in a position to be contacted by liquid. Furthermore, as the superabsorbent material absorbs the liquid it must be allowed to swell. If the superabsorbent material is prevented from swelling, it will cease absorbing liquids.

Adequate absorbency of liquid by the absorbent core at the point of initial liquid contact and rapid distribution of liquid away from this point is necessary to ensure that the absorbent core has sufficient capacity to absorb subsequently deposited liquids. Previously known absorbent cores have thus attempted to absorb quickly and distribute large quantities of liquids throughout the absorbent core while minimizing gel blocking during absorption of multiple doses of liquid.

In general, some of the important performance attributes of an absorbent core of a diaper (or any other absorbent garment) are functional capacity, rate of absorption, core stability in use, type of SAP, ratio of fibrous material to SAP, the type and basis weight of glue or tackifying agent used to adhere the SAP to the fibrous material or tissue wrapping, and the basis weight of the core. Absorption under load or AUL is a good measure of functional capacity and the rate at which that absorption occurs. AUL is believed to be a function of both SAP basis weight (mass per unit area) and the composition of SAP used in the composite. Increasing the basis weight decreases the performance/cost ratio of the absorbent core, making them uneconomical. Also, increased basis weights tend to affect the fit and comfort of the garment, as well as impacting the packaging and shipping costs.

It is known to provide absorbent multi-layer cores comprised of, for example, an upper layer, a lower layer and a central absorbent layer containing from 50% to 95% by weight SAP. U.S. Pat. No. 6,068,620, discloses that the upper and lower layers are comprised of tissue, airlaid fluff pulp or synthetic non-woven fibrous layers. The upper and lower layers are said to assist in maintaining the integrity of the core, the layered arrangement is said to minimize gel blocking, and the multi-layer absorbent composite can be folded in various configurations. It also is known to provide a composite absorbent structure having a wicking layer bonded to the absorbent layer with a bonding agent such that the absorbent structure has a Contact Intimacy Ratio. U.S. Pat. No. 6,239,565 discloses an absorbent composite having a wicking layer that has a vertical wicking flux value, an absorbent liquid retention layer, and a bonding agent.

It is also known to provide an absorbent core having a multiple layers comprising a mixture of fiber and superabsorbent. U.S. Pat. No. 5,728,082 discloses an absorbent body having a first layer of a mixture of fluff and a first superabsorbent, and a second layer having a second superabsorbent having a higher liquid absorbency than the first superabsorbent. U.S. Pat. No. 6,020,536 discloses an absorbent body that includes at least two mutually different cellulose fluffs disposed in two absorbent layers that may also contain superabsorbent materials. U.S. Pat. No. 5,741,241 discloses an absorbent body having a first fiber-based absorbent layer comprised of mixture of cellulose fluff pulp and SAP, a second fiber-based absorbent layer comprised of layers of cellulose fluff pulp and SAP.

### SUMMARY OF THE INVENTION

It would be desirable to provide an absorbent garment having tow fibers, and an increased amount of superabsorbent polymers, particularly with an absorbent core having a reduced amount of fibers, while at the same time having a fast absorbency rate and a high absorbency under load (AUL).

It is therefore a feature of an embodiment of the invention to provide an absorbent garment having an improved ability to absorb and retain fluids, especially in the areas of the core where fluid retention is needed most. It is an additional feature of an embodiment of the invention to provide an absorbent garment that includes an absorbent core having tow fibers and SAP particles, where the SAP particles form a substantial percentage of its basis weight. At the same time, the absorbent garment has reduced gel blocking, i.e., retaining high SAP efficiency. An additional feature of the invention is to provide an absorbent article having specific desired properties in select areas of the absorbent core that is relatively inexpensive to manufacture, that provides the improved properties above, and that is comfortable to wear.

These and other features of the invention can be achieved by an absorbent article including a top sheet, a back sheet and an absorbent core disposed between the top sheet and back sheet. In one embodiment of the invention, the absorbent core of the invention preferably has a central fibrous layer comprising synthetic fibers, an upper layer disposed above the central fibrous layer and comprising a first superabsorbent polymer in free form, and a lower layer disposed below the central fibrous layer and comprising a substantially continuous layer of a second superabsorbent polymer. In this embodiment, the first superabsorbent polymer has different absorbent properties than the second superabsorbent polymer. The upper and/or lower layers may comprise a virgin (unblended) superabsorbent polymer, or they may comprise a blend of two or more superabsorbent polymers.

It is a feature of another embodiment of the invention that the absorbent core preferably has a central fibrous layer containing synthetic fibers, and a lower layer disposed below the central fibrous layer comprising a substantially continuous layer of superabsorbent polymer. The lower layer includes a blend of a first superabsorbent polymer and a second absorbent polymer, where the first superabsorbent polymer has different absorbent properties than the second superabsorbent polymer.

It is a feature of another embodiment of the invention that the absorbent core preferably has a central fibrous layer containing synthetic fibers, an upper layer disposed above the central fibrous layer, comprising a substantially continuous layer of superabsorbent polymer. The upper layer includes a blend of a first superabsorbent polymer and a second absorbent polymer, where the first superabsorbent polymer has different absorbent properties than the second superabsorbent polymer.

In accordance with an additional embodiment of the invention, there is provided a method of making an absorbent article that includes providing a top sheet material and a back sheet material. The method also includes preparing an absorbent core by providing a central fibrous layer having tow fiber, providing an upper layer comprised of a first superabsorbent polymer, and providing a lower layer comprised of a second superabsorbent polymer. The absorbent core is then disposed between the top sheet material and the back sheet material.

These and other features and advantages of the preferred embodiments will become more readily apparent when the detailed description of the preferred embodiments is read in conjunction with the attached drawings.

### BRIEF DESCRIPTION OF THE DRAWINGS

FIG. 1 is a partially cut-away view of an embodiment of the present invention, shown with the elastic members fully stretched in the main portion of the garment;

FIG. 2 is a cross-sectional view of the absorbent garment in FIG. 1 taken along line 2-2;

FIG. 3a is a cross-sectional view of an absorbent core in accordance with an embodiment of the invention;

FIG. 3b is a cross-sectional view of an absorbent core in accordance with an embodiment of the invention;

FIG. 4 is a cross-sectional view of an absorbent core in accordance with an embodiment of the invention;

FIG. 5 is a cross-sectional view of an absorbent core in accordance with an embodiment of the invention;

FIG. 6 is an illustration of an apparatus useful in carrying out a method of making an absorbent garment in accordance with the present invention; and

FIG. 7 is an illustration of an apparatus useful in carrying out a method of making an absorbent garment in accordance with the present invention.

### DETAILED DESCRIPTION OF PREFERRED EMBODIMENTS

As used herein, the terms “absorbent garment,” “absorbent article” or simply “article” or “garment” refer to devices that absorb and contain body fluids and other body exudates. More specifically, these terms refer to garments that are placed against or in proximity to the body of a wearer to absorb and contain the various exudates discharged from the body. A non-exhaustive list of examples of absorbent garments includes diapers, diaper covers, disposable diapers, training pants, feminine hygiene products and adult incontinence products. Such garments may be intended to be discarded or partially discarded after a single use (“disposable” garments). Such garments may comprise essentially a single inseparable structure (“unitary” garments), or they may comprise replaceable inserts or other interchangeable parts.

The present invention may be used with all of the foregoing classes of absorbent garments, without limitation, whether disposable or otherwise. The embodiments described herein provide, as an exemplary structure, a diaper for an infant, however this is not intended to limit the claimed invention. The invention will be understood to encompass, without limitation, all classes and types of absorbent garments, including those described herein. Preferably, the absorbent core is thin in order to improve the comfort and appearance of a garment.

Throughout this description, the expressions “upper layer,” “lower layer,” “above” and “below,” which refer to the various components included in the absorbent core units of the invention (including the layers surrounding the absorbent core units) are used merely to describe the spatial relationship between the respective components. The upper layer or component “above” the other component need not always remain vertically above the core or component, and the lower layer or component “below” the other component need not always remain vertically below the core or component. Indeed, embodiments of the invention include various configurations whereby the core is folded in such a manner that the upper layer ultimately becomes the vertically highest and vertically lowest layer at the same time. Other configurations are contemplated within the context of the present invention. In addition, upper and lower layer refers to the ultimate configuration of the absorbent core, a preferred cross-section of which is illustrated in FIG. 2.

The term “component” can refer, but is not limited, to designated selected regions, such as edges, corners, sides or the like; structural members, such as elastic strips, absorbent pads, stretchable layers or panels, layers of material, or the like; or a graphic.

Throughout this description, the term “disposed” and the expressions “disposed on,” “disposing on,” “disposed in,” “disposed between” and variations thereof (e.g., a description of the article being “disposed” is interposed between the words “disposed” and “on”) are intended to mean that one element can be integral with another element, or that one element can be a separate structure bonded to or placed with or placed near another element. Thus, a component that is “disposed on” an element of the absorbent garment can be formed or applied directly or indirectly to a surface of the element, formed or applied between layers of a multiple layer element, formed or applied to a substrate that is placed with or near the element, formed or applied within a layer of the element or another substrate, or other variations or combinations thereof.

Throughout this description, the terms “top sheet” and “back sheet” denote the relationship of these materials or layers with respect to the absorbent core. It is understood that additional layers may be present between the absorbent core and the top sheet and back sheet, and that additional layers and other materials may be present on the side opposite the absorbent core from either the top sheet or the back sheet.

Throughout this description, the expression “tow fibers” relates in general to any substantially continuous fiber. Tow fibers typically are used in the manufacture of staple fibers, and preferably are comprised of natural and/or synthetic thermoplastic polymers. Usually, numerous filaments are produced by melt extrusion of the molten polymer through a multi-orifice spinneret during manufacture of staple fibers from synthetic thermoplastic polymers in order that reasonably high productivity may be achieved. The groups of filaments from a plurality of spinnerets typically are combined into a tow which is then subjected to a drawing operation to impart the desired physical properties to the filaments comprising the tow. Tow as used in the context of the present invention also encompasses modified tow fibers that have been either surface or internally modified (chemically or otherwise) to improve various desired properties of the fibers (e.g., wicking, etc.).

Throughout this description, the expression “super absorbent polymer” (“SAP”) or “super absorbent material” refers to any polymeric material that is capable of absorbing large quantities of fluid by forming a hydrated gel. Super absorbent polymers are well-known to those skilled in the art as substantially water-insoluble, absorbent polymeric compositions that are capable of absorbing large amounts of fluid (e.g., 0.9% solution of NaCl in water, or blood) in relation to their weight and forming a hydrogel upon such absorption. Super absorbent polymers also can retain significant amounts of water under moderate pressures. Super absorbent polymers generally fall into three classes, namely, starch graft copolymers, cross-linked carboxymethylcellulose derivatives, and modified hydrophilic polyacrylates. Examples of such absorbent polymers are hydrolyzed starch-acrylonitrile graft copolymer; a neutralized starch-acrylic acid graft copolymer, a saponified acrylic acid ester-vinyl acetate copolymer, a hydrolyzed acrylonitrile copolymer or acrylamide copolymer, a modified cross-linked polyvinyl alcohol, a neutralized self-cross-linking polyacrylic acid, a cross-linked polyacrylate salt, carboxylated cellulose, and a neutralized cross-linked isobutylene-maleic anhydride copolymer.

The present invention relates generally to absorbent articles, and in particular to an absorbent article that contains a top sheet, a back sheet, and an absorbent core disposed at least partially between the top sheet and the back sheet. The absorbent core of the invention preferably is comprised of a central fibrous layer containing continuous or discontinuous fibers and one or more upper and/or lower layers, whereby the upper and/or lower layer(s) are comprised primarily of super absorbent polymer (SAP), and the central fibrous layer is a fibrous structure comprised of fibers and, optionally, SAP. In preferred embodiments, the upper layer comprises a different type of SAP than the lower layer.

The invention also relates in general to a method of making an absorbent article that includes providing a top sheet material and a back sheet material. The method also includes preparing an absorbent core that contains at least one upper layer and/or lower layer comprised primarily of SAP, and at least one central fibrous layer comprised of a fibrous material disposed between the upper and lower layers.

The upper layer and/or lower layer of the absorbent cores of the invention preferably contain 50%-100% by weight particulate or fibrous SAP. Each layer may be formed as a substantially continuous layer of SAP, either in free form or disposed on or bound to a substrate layer. Alternately, the upper and/or lower layer may be comprised of a mixture of fiber and SAP. The SAP in the upper and/or lower layer may be a virgin SAP, that is, comprised of only one type of SAP that is pure and unblended. Alternately, the SAP in the upper and/or lower layer may comprise a mixture or blend of two or more SAPs. For example, in certain embodiments, the upper and/or a lower layer may comprise a blend of SAPs, where each SAP in the SAP blend has a different optimized absorbent property, such as a blend of a high AUL SAP and a fast absorption SAP.

In other embodiments, a combination of blended and virgin SAP layers may be used. For example, in one embodiment, the absorbent core has both an upper and a lower layer, where the upper layer has a virgin SAP and the lower layer has a blended SAP. In yet another embodiment, the absorbent core has both an upper and a lower layer, where the upper layer has a blended SAP, and the lower layer has a virgin SAP. In yet other embodiments of the invention, the absorbent core has multiple upper and/or multiple lower layers. In certain embodiments, each layer has different materials and different absorbent properties. For instance, in one embodiments, the absorbent core comprises a central fibrous layer and two upper layers where each layer comprises a different type of SAP or blend of SAPs.

The central fibrous layer of the present invention generally comprises a fibrous structure. The fibrous component of the central fibrous layer may include discontinuous or continuous fibers, such as tow fiber, or a mixture or blend of both. The fibers may be cellulosic fibers, such as cellulose ester fibers, or may be synthetic fibers, such as polypropylene or polyester fibers; or may be any combination or mixture of fibers. The central fibrous layer may optionally contain SAP. Central fibrous layers of this type are generally known in the art, and exemplary absorbent cores are described in U.S. Pat. No. 6,068,620 and U.S. Pat. No. 5,281,207, both issued to Chmielewski, and U.S. Pat. No. 5,863,288, issued to Baker, the disclosures of each of which are herein incorporated by reference in their entirety and in a manner consistent with this disclosure.

The absorbent core and/or the absorbent article also may include one or more additional components, such as at least one layer selected from an acquisition layer, a distribution layer, an additional fibrous layer containing SAP, a wicking layer, a storage layer, or combinations and fragments of these layers. For example, other non-SAP-containing roll good materials such as latex or thermally bonded airlaid fluff pulp, (e.g., roll good available from Walkisoft, Merfin or Fort James), or synthetic spunbonded, carded, or hydro-entangled non-woven may be positioned above and below the absorbent core. The absorbent core may also contain outer containment layers for SAP containment, and/or other layers designed for optimal wet/dry strength, and liquid acquisition and distribution. The absorbent core also may be comprised of more than one absorbent core unit.

The present invention is premised in part on the discovery that providing two distinct layers made of the same or different types of SAP in an absorbent core provides a more efficient absorbent core by optimizing the overall absorbent performance of the absorbent core. Traditional absorbent cores make it difficult to balance the overall absorbency performance by optimizing multiple absorbent properties of the core. These conventional cores typically were designed with a single basis weight, a single type of SAP, a single type of fiber (e.g., fluff pulp) and a single ratio of fiber to SAP. Typically, the absorbent fluff pulp provides fast absorption properties to the absorbent article, and the SAP provides a high absorbency-under-load (“AUL”). Optimizing of these properties is accomplished by varying amounts and ratios of the pulp and SAP. SAP designers have tried to provide SAPs having other desirable absorbent properties, such as a fast absorption rate, but doing so has been at the expense of the AUL, which is a highly desirable property for an absorbent core for an absorbent article. In contrast, in certain embodiments of the present invention, the absorbent core contains a central fibrous layer and an upper layer and a lower layer comprising primarily SAP, where, for example, the SAP of the upper layer has a high AUL, and the SAP of the lower layer has a fast absorption rate. The resultant absorbent core, therefore, has both properties—high AUL and a fast absorption rate. This also allows the product designer to minimize the amount of fiber used in the absorbent core, and optimize the cost of the absorbent core.

The invention now will be described with reference to the attached drawings illustrating preferred embodiments of the invention. For clarity, features that appear in more than one Figure have the same reference number in each Figure.

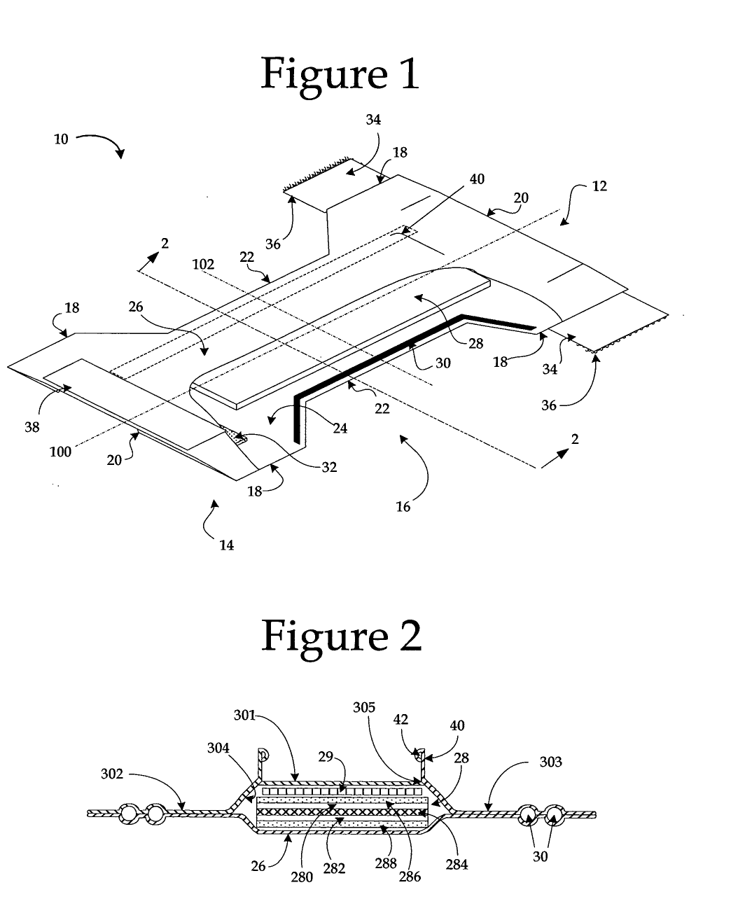

FIG. 1 is a partially cut away depiction of an exemplary embodiment of an absorbent garment 10 (preferably a disposable absorbent garment) of the present invention. The embodiment shown in FIG. 1 is an infant's diaper, however, this depiction is not intended to limit the invention, and those skilled in the art appreciate that the invention covers other types of absorbent articles. For simplicity, however, the invention will be described with reference to an infant's diaper. The garment 10 of FIG. 1 is depicted in a generally flattened position, with the body-facing side facing down, and with the various elastic components depicted in their relaxed condition with the effects of the elastics removed for clarity (when relaxed, the elastics typically cause the surrounding material to gather or “shirr”). In the flattened position, the garment 10 may have a generally hourglass shaped structure, but it may also have any other shape suitable for the given application, such as a rectangular shape, a trapezoidal shape, a “T” shape, and the like.

As used herein, the longitudinal axis 100 of the garment is the dimension of the garment corresponding to the front-to-rear dimension of the user, and the lateral (or transverse) axis 102 of the garment is the dimension corresponding to the side-to-side dimension of the user.

In use, the invention comprises a garment 10 having a pant-like configuration with a waist-encircling region and a crotch region. The waist-encircling region may comprise a first waist region 12, disposed adjacent to, for example, the back waist region of a wearer's body, and a second waist region 14, disposed adjacent to, for example, the front waist region of a wearer's body. The first and second waist regions 12, 14, may correspond to the front and back of the wearer's body, respectively, depending on whether garment 10 is attached in front of or behind the subject wearer. The first and second waist regions are joined together at or near their lateral edges 18, causing the longitudinally distal edges 20 of the garment 10 to form the perimeter of a waist opening. A crotch region 16 extends between the first and second waist regions, 12, 14, and the crotch edges 22 form the perimeter of a pair of leg openings, when the garment 10 is placed on a subject wearer.

The garment 10 preferably comprises a top sheet 24, and a back sheet 26, which may be substantially coterminous with the top sheet 24. When the garment 10 is being worn, the top sheet 24 faces the wearer's body, and the back sheet 26 faces away from the wearer. An absorbent core 28 preferably is disposed between at least a portion of the top sheet 24 the back sheet 26.

An embodiment of the present invention may further comprise various additional features. One or more pairs of elastic gathers 30 (leg elastics) may extend adjacent the crotch edges 22. The garment 10 may also comprise one or more waste containment systems, such as inboard standing leg gathers 40, which preferably extend from the second waist region 14 to the first waist region 12 along opposite sides of longitudinal center line 100 (only one standing leg gather system 40 is shown in FIG. 1 for purposes of clarity). One or both of the first and second waist regions 12, 14 may also be equipped with strips of waist elastic material 32, such as elastic waist foam or other elastically extensible material, which help contract the garment around the wearer's waist, providing improved fit and leakage prevention.

The absorbent garment 10 also preferably includes fastening elements to enable attachment of the first waist region 12 to second waist region 14. Fastening elements preferably include a pair of tabs 34 that extend laterally away from opposite lateral edges 18 of the first waist region 12 of the garment 10. The tabs 34 may comprise an elastically extensible material (not shown), and may be designed to stretch around a wearer's waist to provide improved fit, comfort, and leakage protection. Such tabs 34 may be used in conjunction with, or in lieu of, waist elastic material 32, such as foam, or other elastically extensible materials.

At least one fastening mechanism 36 (collectively referred to as “fastener 36”) is attached to each tab 34 for attaching the tab to the second waist region 14, thereby providing the garment 10 with a pant-like shape, and enabling garment 10 to be fixed or otherwise fitted on the wearer. The fasteners 36 may attach to one or more target devices 38 located in the second waist region 14. For example, in one embodiment of the invention, the fastening mechanism is a hook and loop fastener, where one fastening element is a hook portion, and a corresponding target device is a loop portion of the hook and loop fastener. In another embodiment, the fastening mechanism is a tape fastener system, where one fastening element is an adhesive tape, and a corresponding target device is a tape receiving surface. Other fastening systems may be used in this invention, as long as they are capable of fastening the garment 10 about the wearer.

Although not shown in the drawings, the absorbent garment 10 may also include grips attached along the distal edges of each tab 34 to enable a caregiver to pull the grips, and not on the ends of the tabs 34, around the wearer and over the target devices 38 to thereby secure the fasteners 36 to the one or more target devices 38.

The various parts of the garment 10 can be attached to one another or associated with one another to form a structure that preferably maintains its shape during the useful life of the garment 10. As used herein, the terms “attached,” “joined,” “associated,” and similar terms encompass configurations whereby a first part is directly joined to a second part by affixing the first part directly to the second part, by indirectly joining the first part to the second part through intermediate members, and by fixing the relative positions of various parts by capturing parts between other parts. Those skilled in the art will appreciate that various methods or combinations of methods may be used to securely join the respective parts of the garment 10 to one another.

The top sheet 24 and back sheet 26 may be constructed from a wide variety of materials known in the art. The invention is not intended to be limited to any specific materials for these components. The top sheet 24 and back sheet can be shaped and sized according to the requirements of each of the various types of absorbent garment, or to accommodate various user-sizes. In an embodiment of the invention in which the garment 10 is a diaper or an adult incontinence brief, the combination of top sheet 24 and back sheet 26, may have an hourglass shape, as seen in FIG. 1, or may have a rectangular, trapezoidal, “T” shape, or other shape.

Due to the wide variety of backing and liner sheet construction and materials currently available, the invention is not intended to be limited to any specific materials or constructions of these components. The back sheet 26 preferably is made from any suitable pliable liquid-impervious material known in the art. Typical back sheet materials include films of polyethylene, polypropylene, polyester, nylon, and polyvinyl chloride and blends of these materials. For example, the back sheet can be made of a polyethylene film having a thickness in the range of 0.02-0.04 mm. The back sheet 26 may be pigmented with, for example, titanium dioxide, to provide the garment 10 with a pleasing color or to render the back sheet 26 opaque enough that exudates being contained by the garment 10 are not visible from outside the garment. In addition, the back sheet 26 may be formed in such a manner that it is opaque, for example, by using various inert components in the polymeric film and then biaxially stretching the film. Other back sheet materials will be readily apparent to those skilled in the art. The back sheet 26 preferably has sufficient liquid imperviousness to prevent any leakage of fluids. The required level of liquid imperviousness may vary between different locations on the garment 10.

The back sheet 26 may further comprise separate regions having different properties. In a preferred embodiment, portions of the back sheet 26 are air-permeable to improve the breathability, and therefore comfort, of the garment 10. The different regions may be formed by making the back sheet 26 a composite of different sheet materials, chemical treatment, heat treatment, or other processes or methods known in the art. Some regions of the back sheet 26 may be fluid pervious. In one embodiment of the invention, the back sheet 26 is fluid impervious in the crotch 16, but is fluid pervious in portions of the first and second waist regions 12, 14. The back sheet 26 may also be made from a laminate of overlaid sheets of material.

The moisture-pervious top sheet 24 can be comprised of any suitable relatively liquid-pervious material known in the art that permits passage of liquid there through. Non-woven liner sheet materials are exemplary because such materials readily allow the passage of liquids to the underlying absorbent core 28. Examples of suitable liner sheet materials include non-woven spun bond or carded webs of polypropylene, polyethylene, nylon, polyester and blends of these materials.

The back sheet 26 may be covered with a fibrous, non woven fabric such as is disclosed, for example, in U.S. Pat. No. 4,646,362 issued to Heran et al., the disclosure of which is hereby incorporated by reference in its entirety and in a manner consistent with this disclosure. Materials for such a fibrous outer liner include a spun-bonded non woven web of synthetic fibers such as polypropylene, polyethylene or polyester fibers; a non woven web of cellulosic fibers, textile fibers such as rayon fibers, cotton and the like, or a blend of cellulosic and textile fibers; a spun-bonded non woven web of synthetic fibers such as polypropylene; polyethylene or polyester fibers mixed with cellulosic, pulp fibers, or textile fibers; or melt blown thermoplastic fibers, such as macro fibers or micro fibers of polypropylene, polyethylene, polyester or other thermoplastic materials or mixtures of such thermoplastic macro fibers or micro fibers with cellulosic, pulp or textile fibers. Alternatively, the back sheet 26 may comprise three panels wherein a central poly back sheet panel is positioned closest to absorbent core 28 while outboard non-woven breathable side back sheet panels are attached to the side edges of the central poly back sheet panel. Alternatively, the back sheet 26 may be formed from microporous poly coverstock for added breathability.

As illustrated in more detail in FIG. 2, the top sheet 24 may be formed of three separate portions or panels. Those skilled in the art will recognize, however, that top sheet 24 need not be made of three separate panels, and that it may be comprised of one unitary item. A first top sheet panel 301 may comprise a central top sheet panel formed from preferably a liquid-pervious material that is either hydrophobic or hydrophilic or a combination of the two (i.e., zoned). The central top sheet panel 301 may be made from any number of materials, including synthetic fibers (e.g., polypropylene or polyester fibers), natural fibers (e.g., wood or cellulose), apertured plastic films, reticulated foams and porous foams to name a few. One preferred material for a central top sheet panel 301 is a cover stock of single ply non-woven material which may be made of carded fibers, either adhesively or thermally bonded, perforated plastic film, spunbonded fibers, or water entangled fibers, which generally weigh from 0.3-0.7 oz./sq. yd. and have appropriate and effective machine direction and cross-machine direction strength suitable for use as a baby diaper cover stock material. The central top sheet 301 panel preferably extends from substantially the second waist region 14 to the first waist region 12, or a portion thereof.

The second and third top sheet panels 302, 303 (e.g., outer top sheet panels), in this alternative embodiment may be positioned laterally outside of the central top sheet panel 301. The outer top sheet panels 302, 303 are preferably substantially liquid-impervious and hydrophobic, preferably at least in the crotch area. The outer edges of the outer top sheet panels may substantially follow the. corresponding outer perimeter of the back sheet 26. The material for the outer top sheet portions or panels is preferably polypropylene and can be woven, non-woven, spunbonded, carded or the like, depending on the application.

The inner edges 304 (FIG. 2) of the outer top sheet portions or panels 302, 303 preferably are attached by, e.g., an adhesive, to the outer edges 305 of the inner top sheet portion or panel 301. At the point of connection with the outer edges 305 of the inner top sheet portion or panel 301, the inner edges 304 of the outer top sheet portions or panels 302, 303 extend upwardly to form waste containment flaps 40. The waste containment flaps 40 preferably are formed of the same material as the outer top sheet portions or panels 302, 303, as in the embodiment shown. They are preferably an extension of the outer top sheet portions or panels 302, 303.

The waste containment flaps 40 may be treated with a suitable surfactant to modify their hydrophobicity/hydrophilicity as desired, and they may be treated with skin wellness ingredients to reduce skin irritation. Alternatively, the waste containment flaps 40 may be formed as separate elements and then attached to the body side liner. In this alternative embodiment, the central top sheet portion or panel 301 may extend past the connection point with the waste containment flaps 40, and even extend to the periphery of the back sheet 26.

The waste containment flaps 40 preferably include a portion that folds over onto itself to form a small enclosure. At least one, and depending on the size of the enclosure sometimes more than one, elastic member 42 may be secured in the enclosure in a stretched condition. As is known in the art, when the elastic member 42 attempts to assume the relaxed, unstretched condition, the waste containment flaps 40 rise above the surface of the central top sheet portion or panel 301.

The top sheet 24 (as well as top sheet portions 301, 302, 303) may be made of any suitable relatively liquid-pervious material currently known in the art or later discovered that permits passage of a liquid there through. Examples of suitable top sheet materials include non-woven spun-bonded or carded webs of polypropylene, polyethylene, nylon, polyester and blends of these materials, perforated, apertured, or reticulated films, and the like. Non-woven materials are exemplary because such materials readily allow the passage of liquids to the underlying absorbent laminate core 28. The top sheet 24 preferably comprises a single-ply non-woven material that may be made of carded fibers, either adhesively or thermally bonded, spun-bonded fibers, or water entangled fibers, which generally weigh from 0.3-0.7 oz./sq. yd. and have appropriate and effective machine direction (longitudinal) and cross-machine (lateral) direction strength suitable for use as a top sheet material for the given application. The present invention is not intended to be limited to any particular material for the top sheet 24, and other top sheet materials will be readily apparent to those skilled in the art.

The top sheet 24 may further comprise several regions having different properties. In one embodiment of the present invention, the laterally distal portions of the top sheet 24, especially those used to make second and third top sheet panels 302, 303, preferably are substantially fluid impervious and hydrophobic, while the remainder of the top sheet 24 (e.g., central top sheet panel 301) is hydrophilic and fluid pervious. Different top sheet properties, such as fluid perviousness and hydrophobicity, may be imparted upon the top sheet 24 by treating the top sheet 24 with adhesives, surfactants, or other chemicals, using a composite of different materials, or by other means. The top sheet 24 may also be made from a laminate of overlaid sheets of material. The top sheet 24 also may be treated in specific areas like the crotch region, with skin wellness ingredients such as aloe, vitamin E, and the like.

As noted elsewhere herein, the top sheet 24 and back sheet 26 may be substantially coterminous, or they may have different shapes and sizes. The particular design of the top sheet 24 and back sheet 26 may be dictated by manufacturing considerations, cost considerations, and performance considerations. Preferably, the top sheet 24 is large enough to completely cover the absorbent core 28, and the back sheet 26 is large enough to prevent leakage from the garment 10. The design of top sheet 24 and back sheet 26 is known in the art, and a person of ordinary skill in the art will be able to produce an appropriate top sheet 24 and an appropriate back sheet 26 without undue experimentation.

The top sheet 24 and the back sheet 26 may be associated with one another using a variety of methods known in the art. For example, they may be thermally, ultrasonically, or chemically bonded to one another. They also may be joined using a hot melt adhesive or mechanical fasteners, such as thread, clips, or staples. In one embodiment, a hydrophilic adhesive, such as CYCLOFLEX, sold by National Starch and Chemical Company, a corporation headquartered in Bridgewater, N.J., is used to join the top sheet 24 to the back sheet 26. The particular joining method may be dictated by the types of materials selected for the top sheet 24 and back sheet 26.

As mentioned above, absorbent garment preferably is provided with leg elastics 30 extending through crotch region 16, adjacent crotch edge 22. The absorbent garment of the invention also preferably is provided with waist elastic material 32 optionally in the first and second waist regions; 12, 14, respectively, to enable and assist in stretching around the wearer. The waist elastic materials 32 may be similar structures or different to impart similar or different elastic characteristics to the first and second waist regions 12, 14 of the garment. In general, the waist elastic materials may preferably comprise foam strips positioned at the first and second waist regions 12, 14, respectively. Such foam strips preferably are about ½ to about 1½ inches wide and about 3-6 inches long. The foam strips preferably are positioned between the top sheet 24 (or panels 301, 302, 303) and the back sheet 26. Alternatively, a plurality of elastic strands may be employed as waist elastics rather than foam strips. The foam strips preferably are comprised of polyurethane, but can be any other suitable material that decreases waist band roll over, reduces leakage over the waist ends of the absorbent garment, and generally improve comfort and fit. The first and optional second waist foam strips preferably are stretched 50-150%, preferably 100% more than their unstretched dimension before being adhesively secured between the back sheet 26 and top sheet 24.

Each edge 22 that forms the leg openings preferably is provided with adjacent leg elastics 30 to form a containment system. In the preferred embodiment, three strands of elastic threads (only two strands are shown in FIG. 2 for purposes of clarity) are positioned to extend adjacent to leg openings between the outer top sheet portions or panels 302, 303 and the back sheet 26. Any suitable elastomeric material exhibiting at least an elongation (defined herein as (L_{S}-L_{R})/L_{R} where L_{S} is the stretch length of an elastic element and L_{R} is retracted length, multiplied by 100 to obtain percent elongation) in the range of 5%-350%, preferably in the range of 200%-300%, can be employed for the leg elastics 30. The leg elastics 30 may be attached to the absorbent article 10 in any of several ways which are known in the art. For example, the leg elastics 30 may be ultrasonically bonded, heat/pressure sealed using a variety of bonding patterns, or glued to the garment 10. Various commercially available materials can be used for the leg elastics 30, such as natural rubber, butyl rubber or other synthetic rubber, urethane, elastomeric materials such as LYCRA (INVISTA, Inc., Wilmington, Del.), S-72 (Radici Spandex, Fall River, Mass.) or SYSTEM 7000 (Fulflex, Inc., Lincoln, R.I.).

The fastening elements, preferably a fastening system 34 (e.g., tab 34) of the preferred embodiment, is attached to the first waist region 12, and it preferably comprises a tape tab or mechanical fasteners 36. However, any fastening mechanism known in the art will be acceptable. Moreover, the fastening system 34 may include a reinforcement patch below the front waist portion so that the diaper may be checked for soiling without compromising the ability to-reuse the fastener. Alternatively, other absorbent article fastening systems are also possible, including safety pins, buttons, and snaps.

As stated previously, the invention has been described in connection with a diaper. The invention, however, is not intended to be limited to application only in diapers. Specifically, the absorbent cores of the preferred embodiments may be readily adapted for use in other absorbent garments besides diapers, including, but not limited to, training pants, feminine hygiene products and adult incontinence products.

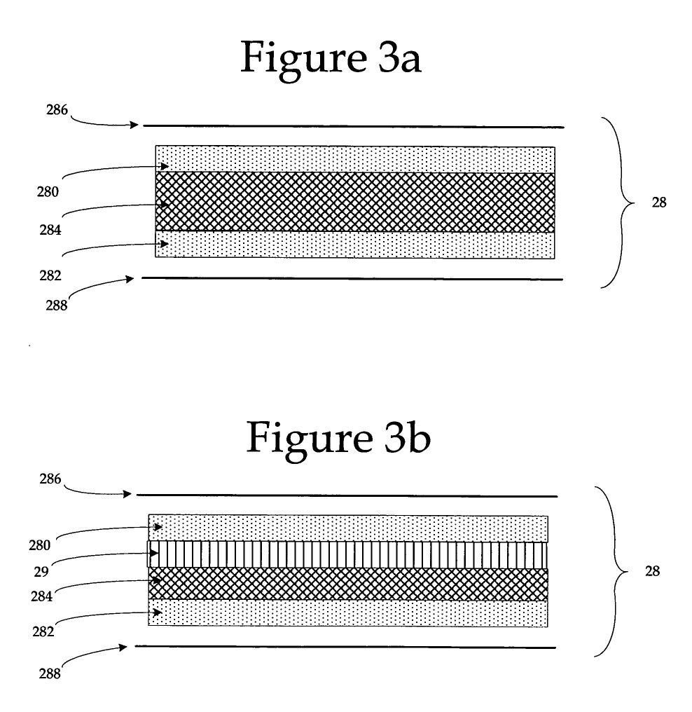

The underlying structure beneath the top sheet 24 may include, depending on the diaper construction, various combinations of elements, but in each embodiment, it is contemplated that the absorbent garment will preferably include a absorbent core 28 comprising multiple layers positioned between the top sheet 24 and back sheet 26. In addition, an additional layer 29 may be disposed between the top sheet 24 and absorbent core 28, and/or other additional layer(s) 29 may be disposed between these layers (e.g., FIG. 3b), or between absorbent core 28 and back sheet 26 (e.g., FIG. 5). The additional layer(s) 29 may include a fluid transfer layer, a fluid handling layer, a storage layer, a wicking layer, a fluid distribution layer, and any other layer(s) known to those having ordinary skill in the art.

Although the absorbent core 28 depicted in FIG. 1 has a substantially rectangular cross-sectional and plan view shape, other shapes may be used, such as a “T” shape or an hourglass shape. The shape of the absorbent core 28 may be selected to provide the greatest absorbency with a reduced amount of material. The absorbent core may be associated with the top sheet 24, back sheet 26, or any other suitable part of the garment 10 by any method known in the art, in order to fix the absorbent core 28 in place. In addition to the respective layers in the absorbent core 28, the overall absorbent core 28 may be enclosed within a tissue wrapping, as disclosed in U.S. Pat. No. 6,068,620, the disclosure of which is incorporated by reference herein in its entirety. Persons of ordinary skill in the art are capable of designing and wrapping a suitable absorbent core 28 of the invention, using the guidelines provided herein.

The absorbent core 28 may extend into either or both of the first and second waist regions 12, 14. The absorbent core 28 of one preferred embodiment of the invention preferably includes at least two (2) layers whereby one of the layers is a central fibrous layer 284, preferably comprising substantially continuous or discontinuous synthetic fibers, and the other layer is either an upper layer 280 or a lower layer 282, containing primarily SAP. In another preferred embodiment of the invention, the absorbent core 28 includes at least three (3) layers—a central fibrous layer 284 comprised of synthetic fibers, an upper layer 280 and a lower layer 282, both containing primarily SAP.

In a preferred embodiment, the central fibrous layer 284 of absorbent core 28 comprises a fibrous structure. The central fibrous layer 284 comprises substantially continuous or discontinuous fibers, or a mixture or combination thereof. Preferably, the central fibrous layer 284 comprises a substantially continuous tow fiber, and more preferably, a substantially continuous crimped-filament tow fiber. A substantially continuous fiber structure has high structural integrity, and as such, is distinct from a matrix of fibers such as fluff, or fluff pulp. The high structural integrity of substantially continuous-fiber matrices enables the production of stronger webs. This, in turn, enables the production of thinner absorbent pads. In addition, the use of such fibers enables the production of ultra low density absorbent cores, when compared to traditional absorbent cores prepared by dispersing SAP particles in fluff pulp fibers.

The tow fiber can be any substantially continuous thermoplastic filament tow fiber that is capable of being opened and used in an absorbent core, alone or in combination with SAP. Before making an absorbent core that includes a tow fiber, the tow fiber typically is unwound and opened, and then fed to the core forming station to provide a fibrous mass of material. Persons of ordinary skill in the art are aware of techniques available to open tow fibers and form the opened fibers into a fibrous mass. Alternately, the tow fiber may be formed into an absorbent web in a separate process, and provided as a low-density roll-good material to form the central fibrous layer 284. Still further yet, the fibrous component could be a carded web formed on-line.

Fibrous additives of central fibrous layer 284 preferably include, but are not limited to, synthetic fibers, such as cellulose ester fibers, cellulose acetate fibers, rayon fibers, lyocell fibers, polyolefin fibers, polyacrylonitrile fibers, surface-modified (hydrophilic) polyester fibers, surface-modified polyolefin/polyester bicomponent fibers, surface-modified polyester/polyester bicomponent fibers, or natural fibers, such as cotton or cotton linter fibers, or blends thereof. The fibrous additives are preferably synthetic fibers. Of the foregoing, cellulose acetate is the most preferred synthetic fibrous additive for use in central fibrous layer 284. In addition, rayon, lyocell and polyacrylonitrile fibers have similar properties to cellulose acetate and are alternatively preferred. The remaining synthetic fibers, polyolefin fibers, surface-modified polyolefin/polyester bicomponent fibers, and surface-modified polyester/polyester bicomponent fibers are also believed to be effective fibrous additives.

Preferably, polypropylene or cellulose ester tow is used as the fibrous material in central fibrous layer 284. Non-limiting examples of suitable cellulose esters include cellulose acetate, cellulose propionate, cellulose butyrate, cellulose caproate, cellulose caprylate, cellulose stearate, highly acetylated derivatives thereof such as cellulose diacetate, cellulose triacetate and cellulose tricaproate, and mixtures thereof such as cellulose acetate butyrate. A suitable cellulose ester will include some ability to absorb moisture, (but absorptive capacity is not necessarily required), preferably is biodegradable, and is influenced not only by the substituent groups but also by the degree of substitution. The relationship between substituent groups, degree of substitution and biodegradability is discussed in W. G. Glasser et al., BIOTECHNOLOGY PROGRESS, Vol. 10, pp. 214-219 (1994), the disclosure of which is incorporated herein by reference in its entirety.

The substantially continuous filament tow useful in the present invention is beneficially moisture-absorbent and biodegradable. Accordingly, cellulose acetate tow is typically preferred for use in the invention. Typically, the denier per fiber (dpf) of the tow fiber will be in the range of about 1 to 25, preferably about 3 to 15, and most preferably about 6 to 7. For the same weight product, filaments of lower dpf may provide increased surface area and increased moisture absorption. Total denier may vary within the range of about 5,000 to 60,000, more preferably from about 20,000 to about 40,000, and most preferably from about 20,000 to about 30,000, depending upon the process used.

It is particularly preferred in the invention to use tow having crimped filaments. Tow materials having crimped filaments are typically easier to open. Separation of filaments resulting from bloom advantageously results in increased available filament surface area for superabsorbent material immobilization and increased moisture absorption. Gel blocking also may be reduced by using-crimped tow in the central fibrous layer 284. As therefore may be understood, more crimp is typically better, with in excess of about 20 crimps per inch being usually preferred. Substantially continuous filament, cellulose ester tow having crimped filaments with about 25 to 40 crimps per inch, is commercially available from Celanese Acetate, Charlotte, N.C.

It is preferred in the present invention that the tow fibers in central fibrous layer 284 have an average length generally about the same length as the absorbent core 28. Typically, the tow is a substantially continuous filament that is cut to length during manufacture of the absorbent core. In comparison, discontinuous fibers have an average fiber length that is somewhat shorter than the length of the absorbent core 28. The discontinuous fibers may be cut to length either during manufacture of the absorbent core 28, or during a separate process, such as the manufacture of a roll-good material. The average diameter of the tow fibers typically is expressed as the cross sectional area of the fibers, although the width of the fibers preferably is within the range of from about 50 to about 200 mm, more preferably from about 75 to about 150 mm, and most preferably from about 85 to about 120 mm. The cross sectional area is based on the denier and density of the fibers. For example, the denier per foot (dpf) and density (typically an acetate polymer density is about 1.32 g/cm³), can be used to calculate the cross sectional area. A 3.0 dpf acetate polymer fiber has a cross sectional area 2.525×10⁻⁶ cm².

The basis weight of preferred fibers used in the central fibrous layer 284 ranges from about 20 to about 200 g/m², more preferably from about 40 to about 120 g/m², and most preferably from about 60 to about 90 g/m².

Optionally, it is advantageous to introduce from about 1-5% of a thermally bondable fiber into the fibrous component of the central fibrous layer 284 for wet strength and core stability in use. In addition to the tow material used as the fibrous component in central fibrous layer 284, other fibrous components also may be used. For example, additional tow fibers (different from original two fiber) or a low-density roll good made in a separate process may be used in central fibrous layer 284. Alternately, the fibrous component could include a carded web formed on-line.

If desired, an absorptive pad of multiple layer thickness, may be provided. To this end, the tow may be, for example, lapped or crosslapped in accordance with conventional procedures. In this way, an absorptive material of a desired weight and/or thickness may be provided. The specific weight or thickness will depend upon factors including the particular end use.

The central fibrous layer 284 may optionally contain SAP. Any superabsorbent polymer (SAP) now known or later discovered may be used in the central fibrous layer 284 so long as it is capable of absorbing liquids. Useful SAP materials are those that generally are water-insoluble but water-swellable polymeric substance capable of absorbing water in an amount that is at least ten times the weight of the substance in its dry form. Super absorbent polymers also can retain significant amounts of water under moderate pressures.

In one type of SAP, the particles or fibers may be described chemically as having a back bone of natural or synthetic polymers with hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Such modified polymers may also be cross-linked to reduce their water-solubility.

Examples of suitable SAPs are water swellable polymers of water soluble acrylic or vinyl monomers crosslinked with a polyfunctional reactant. Also included are starch modified polyacrylic acids and hydrolyzed polyacrylonitrile and their alkali metal salts. A more detailed recitation of superabsorbent polymers is found in U.S. Pat. No. 4,990,541 to Nielsen, the disclosure of which is incorporated herein by reference in its entirety.

Commercially available SAPs include a starch modified superabsorbent polymer available under the trade name HYSORB from BASF Aktiengesellschaft, Ludwigshafen, Germany. Other commercially available SAPs include a superabsorbent derived from polypropenoic acid, available under the tradename DRYTECH 520 SUPERABSORBENT POLYMER from The Dow Chemical Company, Midland Mich.; AQUA KEEP, and AQUA KEEP SA60S, manufactured by Sumitomo Seika Chemicals Co., Ltd., Osaka Japan.; ARASORB manufactured by Arakawa Chemical (U.S.A.) Inc.; FAVOR manufactured by Stockhausen, Inc.; DIAWET, commercially available from Mitsubishi Chemicals, Japan; FLOSORB, available from SNF Floerger, France, AQUALIC, available from Nippon Shokubai, Osaka, Japan.

The SAP can be in the form of particulate matter, flakes, fibers and the like. Exemplary particulate forms include granules, pulverized particles, spheres, aggregates and agglomerates. The SAP may be provided in any particle size, and suitable particle sizes vary greatly depending on the ultimate properties desired.

It has been known to prepare absorbent cores comprised of cellulose acetate tow or other polymeric fibers and SAP, as described in H1565, and U.S. Pat. Nos. 5,436,066, and 5,350,370, the disclosures of each of which are incorporated by reference herein in its entirety. It was conventional to add fackifying agents, specific size fibers, or specific fibers in combination with fluff, in order to prepare the absorbent core and immobilize the SAP particles.

The concentration of fibrous material in the central layer 284 of the absorbent core 28 preferably is about 40%-100%, more preferably about 60%-100%, and most preferably about 80%-100%. Most preferably, the central fibrous layer 284 comprises from about 0%-20% SAP and from about 80-100% fibrous materials selected from the foregoing group, or the fibrous components discussed below.

Particulate additives may be added to central fibrous layer 284 in addition to or as a substitute for the foregoing fibrous and particulate additives in order to maintain high SAP efficiency. The particulate additives preferably are insoluble, hydrophilic polymers with particle diameters of 100 μm or less. The particulate additives are chosen to impart optimal separation of the SAP particles. Examples of preferred particulate additive materials include, but are not limited to, potato, corn, wheat, and rice starches. Partially cooked or chemically modified (i.e., modifying hydrophobicity/hydrophilicity, softness, and hardness) starches can also be effective. Most preferably, the particulate additives comprise partially cooked corn or wheat starch because in this state, the corn or wheat are rendered larger than uncooked starch and even in the cooked state remain harder than even swollen SAP. In any event, regardless of the particulate additive chosen, one of the many important criteria is to use particulate additives that are hard hydrophilic materials relative to swollen SAP or which are organic or inorganic polymeric materials about 100 microns in diameter. Fibrous and particulate additives can be used together in these absorbent laminates. Examples of SAP/particulate and SAP/fiber/particulate additives include those described in, for example, U.S. Pat. No. 6,068,620.

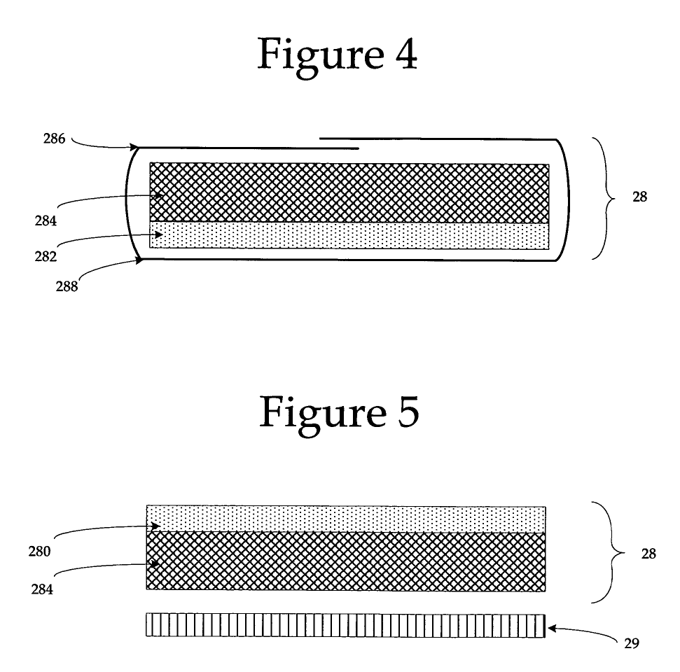

Referring to FIGS. 3a, 3b, 4 and 5, the absorbent core 28 may contain one or more upper layers 280 and/or lower layers 282. For example, FIG. 3a shows an absorbent core configuration having a central fibrous layer 284, an upper layer 280 disposed on the upper surface of the central fibrous layer 284, and a lower layer 282 disposed on the bottom surface of the central fibrous layer 284. FIG. 4 shows an absorbent core 28 having a central fibrous layer 284, with a lower layer 282 disposed on the bottom surface of the central fibrous layer. FIG. 5 shows an absorbent core 28 having a central fibrous layer 284 and an upper layer 280 disposed on the upper surface of the central fibrous layer.

The upper and/or lower layers 280, 282, comprise primarily SAP. Preferably, the upper and/or lower layers 280, 282 contain about 50% to about 100% by weight particulate or fibrous SAP. Any superabsorbent polymer (SAP) now known or later discovered may be used in upper or lower layers 280, 282 so long as it is capable of absorbing liquids. Each layer 280, 282 may be formed as a substantially continuous layer of SAP, or may be comprised of a mixture of fiber and SAP. For example, the layer 280, 282 may include a substrate material, such as a tissue or nonwoven, onto which the SAP is adhered or bound. Alternately, the upper and/or lower layer 280, 282 can be comprised of an airlaid material that contains a mixture of fiber and SAP. The SAP/fiber ratio may be homogeneous throughout the layer, or may vary within the layer. The layer may also contain adhesives, tackifying agents or binders to contain the SAP within the layer. Other fibrous or particulate additives may be used to maintain high SAP efficiencies. Persons of ordinary skill in the art will be capable of identifying or designing such a layer.

In one preferred embodiment, the absorbent core 28 comprises an upper layer 280 that is comprised of free SAP (e.g., SAP that is not bonded or adhered to a substrate layer) and a lower layer 282 disposed below the central fibrous layer 284 that is comprised of a tissue layer onto which a substantially continuous layer of SAP is adhered. In this embodiment, the free SAP that is initially placed on top of the central fibrous layer 284, but may migrate into the fibers of the central fibrous layer after shifting or movement of absorbent core. In another preferred embodiment, the lower layer 282 comprises an airlaid pulp web with which a substantially continuous layer of SAP is combined (e.g., blended or bound to).

The upper and/or lower layers 280, 282 may contain a virgin SAP, that is, comprised of only one type of SAP that is pure and unblended. Alternately, the SAP in the upper and/or lower layer may comprise a mixture or blend of two or more SAPs. For example, in certain embodiments, the upper layer 280 may comprise a virgin SAP, and the lower layer 282 comprises a blend of two or more SAPs. In other embodiments, the lower layer 282 may comprise a virgin SAP, and the upper layer 280 comprises a blend of two or more SAPs. In other embodiments, both the upper layer 280 and lower layer 282 comprise a blend of two or more SAPs, or both comprise virgin SAPs.

In a preferred embodiment of the present invention, the absorbent core 28 contains two or more different types of SAP. In other words, each type of SAP has one or more properties which distinguish it from the one or more other SAPs used in the absorbent article of the present invention. For instance, a SAP type may be defined or distinguished from another type of SAP based on an absorbent property of the SAP, such as absorbency under load (AUL), permeability or absorption rate. Other distinguishing features that may be used to define a SAP type include, for instance, particle size, chemistry, particle geometry, flow agent additives, color, etc.

Preferably, absorbent core 28 comprises two or more types of SAP. This may be achieved, for example, by having an upper layer 280 having a first SAP type, and a lower layer 282 having a second SAP type. Alternately, one or both of the upper and lower layers 280, 282 may contain a blend of two different types of SAP. As a result of having two or more types of SAP, the performance of the absorbent core 28 benefits from the properties and locations of each type of SAP. For instance,.in one preferred embodiment of the present invention, the absorbent core 28 has a central fibrous layer 284, an upper layer 280 comprised of a layer of a free SAP having a high-AUL, and a lower layer 282 comprised of a layer of SAP adhered to a tissue layer, the SAP in the lower layer having fast absorption. The resultant absorbent core 28, has the benefits of fast acquisition of fluid, due to the SAP in the lower layer 282, and low rewet (indication of dryness of the skin-facing surface) because of the high-AUL SAP in the upper layer 280. This combination of properties would be unavailable in a traditional absorbent core comprising fluff pulp fiber and a single SAP. In another exemplary embodiment, the upper layer 280 comprises a blend of high AUL SAP and permeable SAP and the lower layer 282 comprises a blend of fast absorbing SAP and permeable SAP. The resultant absorbent core 28, has the benefits of fast acquisition of fluid, due to the SAP in the lower layer 282, and low rewet (indication of dryness of the skin-facing surface) because of the high-AUL SAP in the upper layer 280, as well as reduced gel-blocking due to the permeable SAP present in both layers. Again, this combination of properties would be unavailable in a traditional absorbent core comprising fluff pulp fiber and a single SAP.

The upper and/or lower layers 280, 282 may have the same overall length and same overall width as the central fibrous layer 284. Alternately, the upper and/or lower layers 280, 282 may have a length that is longer or shorter than the length of the central fibrous layer 284. Likewise, the upper layer and/or lower layers 280, 282 may have a width that is wider or narrower than the width of the central fibrous layer 284. In certain embodiments of the invention, the upper layer and/or lower layer 280, 282 may be fragmented (i.e., discontinuous) either in the longitudinal direction or the transverse direction.

In addition, absorbent core 28 may contain one or more additional layer(s) 29 disposed on or between any of the layers 280, 282, 284. For example, FIG. 3bshows additional layer 29 disposed between upper layer 280 and central fibrous layer 284, alternately the additional layer 29 may be disposed on or between any of the layers of the absorbent core 28. The additional layer(s) 29 may include a fluid transfer layer, a fluid handling layer, a storage layer, a wicking layer, a fluid distribution layer, an additional fibrous layer or any other layer(s) known to those having ordinary skill in the art, or combinations and fragments of these layers. Such layers may be provided to assist with transferring fluids to the absorbent core 28, handling fluid surges, preventing rewet, containing absorbent material, improving core stability, or for other purposes. For example, a wicking layer having enhanced lateral wicking capabilities may be provided in an absorbent core 29 that has enhanced absorbency near its edges. Additional layer 29 may be, for example, a non-SAP-containing roll good material such as latex or thermally bonded airlaid fluff pulp, (e.g., roll good available from Walkisoft, Merfin or Fort James), or synthetic spunbonded, carded, or hydro-entangled non-woven. The absorbent core 28 may also contain additional outer layers designed for optimal wet/dry strength, liquid acquisition and distribution, as well as SAP containment. The absorbent core 28 also may be comprised of more than one absorbent core unit.

Optionally, about 1-10%, preferably about 5%, by weight of thermally bondable synthetic fibers can be added to the absorbent core 28 to impart additional wet strength to the laminate. This will improve the stability of the core during use of the diaper. The preferred synthetic fibers are polyolefin/polyester fibers and polyester/polyester bicomponent fibers.

The foregoing absorbent cores 28 of the preferred embodiments preferably are made using a dry process, whereby the respective components of the composite core 28 are brought together in a dry state, as opposed to one or more components being in a liquid state. Persons of ordinary skill in the art will be capable of making the absorbent cores 28 of the present invention, using the guidelines provided herein.

Optionally, the absorbent core 28 may be at least partially enclosed between upper and lower containment layers 286, 288 (see FIGS. 3a, 3b and 4). Upper and lower containment layers 286, 288 can be made of any suitable material capable of containing the inner layers of the absorbent core 28. Preferably, upper containment layer 286 is hydrophilic and fluid pervious, and lower containment layer 288 is hydrophobic and substantially fluid impervious. It is preferred that upper and lower containment layers 286, 288 are preferably comprised of a material selected from the group consisting of tissue, nonwoven, film and other like structures. In preferred embodiments, the upper and/or lower containment layers 286, 288, are comprised of a tissue or nonwoven substrate. In certain other embodiments, the upper and/or lower containment layers are comprised of a portion of the topsheet, and/or backsheet. The upper and/or lower containment layers 286, 288, may be comprised of the same materials, or of different materials. The upper and lower containment layers 286, 288, preferably are wider than the central fibrous layer 284 and upper and/or lower layers. 280, 282, and their side portions preferably are sealed to one another by bonding, by crimping or by both to prevent release of opened tow and particles of SAP. In another embodiment, as shown in FIG. 4, the upper and lower containment layers 286, 288, are comprised of the same material folded over onto itself, enveloping the absorbent core 28, and only the open edge sealed by crimping or bonding.

The total basis weight of the absorbent core 28 including fibrous materials, SAP, outer layers, additional layers, containment layers and additives, can be anywhere from about 50-1,000 grams per square meter. The most preferred total basis weights of the absorbent core 28 are about 300-700 grams per square meter.

The absorbent core 28 comprising the assembly of the central fibrous layer 284, the upper and/or lower layers 280, 282, and the optional additional layer(s) 29 and containment layers 286, 288, may be further processed as it is conveyed through the assembly line for inclusion into absorbent garments. For example, the absorbent core 28 may be severed into individual absorbent cores, and the severed ends may be crimped or bonded or both to prevent the SAP from exiting the ends.

Crimping, bonding or both can be performed on the absorbent core 28 of the invention using conventional means. For example, the lateral side edges, and longitudinal edges can be sealed together by intermittent or continuous application of adhesive to the respective portions of the upper and lower containment layers 286, 288 using any device capable of applying adhesives to a continuous moving web of material. The lateral and/or longitudinal edges then can be pressed together to form a seal. The seal also can be formed ultrasonically, or the respective edges (lateral and/or longitudinal) can be crimped using crimping rollers or any other crimping device known to those having ordinary skill in the art. Using the guidelines provided herein, those skilled in the art will be capable of sealing the lateral and/or longitudinal edges of absorbent core 28 using bonding, crimping, or both.

It is possible in the present invention that the absorbent core 28 be folded as it is disposed between the top sheet 24 and back sheet 26. The absorbent core 28 can be folded longitudinally, transversely or otherwise, in any suitable manner, including any and all of those disclosed in U.S. Pat. No. 6,068,620. Suitable folds include “C” folds, “G” folds, “U” folds, “A” folds, pleats or “W” folds, and the like.

The invention also relates to a method of making an absorbent core, and an absorbent article that includes providing a top sheet material 24 and a back sheet material 26. The method also includes preparing an absorbent core 28 by disposing a central fibrous layer 284 between an upper layer 280 and/or a lower layer 282, whereby the upper layer 280 and/or lower layer 282 are comprised primarily of SAP. The method includes disposing the absorbent laminate core 28 between the top sheet 24 and the back sheet 26. Preparing the absorbent laminate core 28 includes supplying a tow fiber to provide the central fibrous layer 284, and providing an upper layer 280 and lower layer 282, comprised primarily of SAP. Additional layers 29, may be provided by the process, and disposed either within the absorbent core 28, or between absorbent core 28 and the topsheet and/or backsheet layers. Likewise, containment layers 286, 288, may be provided by the process, and disposed on the upper and/or lower surface of the absorbent core 28.

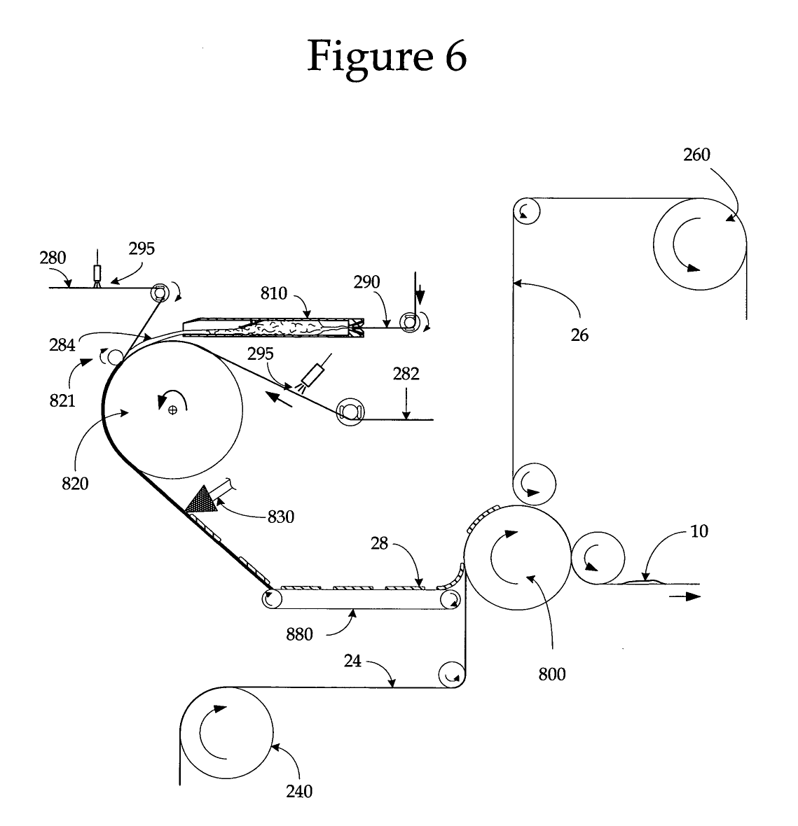

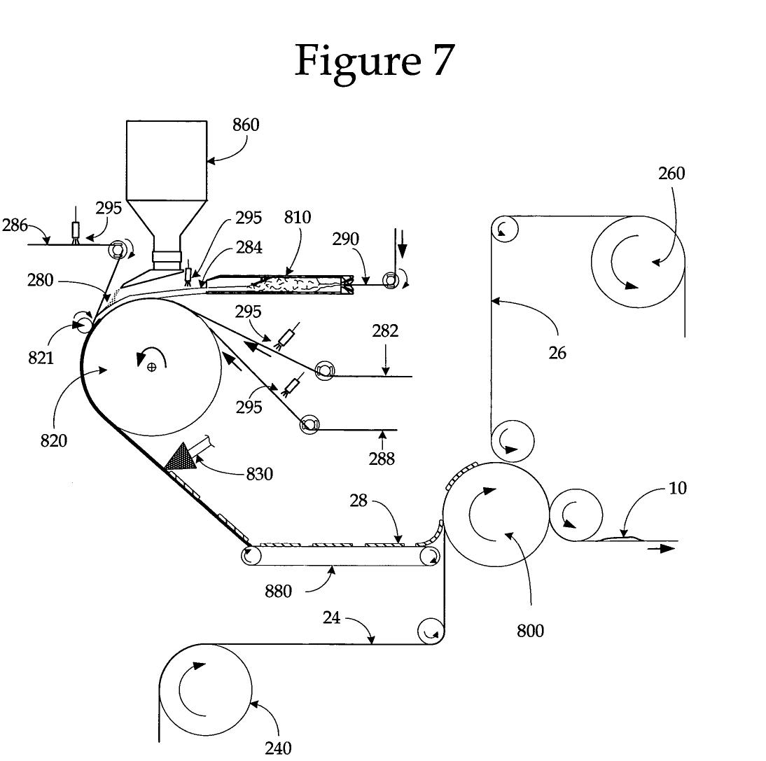

FIGS. 6 and 7 illustrate two exemplary apparatus useful in forming an absorbent article 10 in accordance with particular embodiments of the present invention. Any type of tow fiber 290 can be supplied to the apparatus and, as conventional in the art, the tow fiber 290 typically is opened prior to forming a fibrous matrix. In this regard, the apparatus includes a tow opener and feeder 810 that is capable of opening any suitable tow material, expanding the tow fiber and feeding the tow fiber to the core forming station 820. Any suitable tow opener and feeder 810 can be used in the method of the invention.

Absorbent core 28 can be formed at core forming station 820, where central fibrous layer 284 is disposed between an upper layer 280 and lower layer 282, as shown in FIG. 6. Additional layer(s) 29 and/or containment layer(s) 286, 288 also may be fed to the core forming unit 820, such as shown in FIG. 7. Any or all of the layers may be supplied to the core forming station 820 using any supplying mechanism known in the art, and preferably are fed through one or more feed rollers. Adhesive 295 can be applied to the layers by an adhesive applicator. Again, any mechanism capable of supplying an adhesive 295, albeit a spray adhesive, or one that is “rubbed” on, can be used in the invention. It is particularly preferred to use hot melt construction adhesives, including HL-1258 by H.B. Fuller Company of St. Paul, Minn.; H2587-01 by Ato Findley Inc. of Wauwatosa, Wis.; and NS 34-5665 by National Starch and Chemical Co. of Bridgewater, N.J. The adhesives used in the invention may be used in all adhesive applications in the absorbent garment, or only in select applications as a construction adhesive for bonding parts of the garment as the top sheet, back sheet, absorbent core, and additional layer(s). The positioning and amount of adhesive 295 may be either controlled manually, or controlled by adhesive controller. Any system can be used as adhesive applicator controller to control the amount, if any, and location of the application of the adhesive 295. Those of ordinary skill in the art are capable of designing a suitable adhesive application controller to apply select amounts of adhesive 295, in select positions within or on the layers of the absorbent core 28, using the guidelines provided herein. The multiple layers of absorbent core 28 may then become affixed with the adhesive 295 when the absorbent core 28 is passed through the one or more nip rollers 821 at the core forming station 820.

Referring now specifically to FIG. 7, in providing an upper layer 280 comprising a free SAP layer, the SAP may be fed to the absorbent core 28 by any SAP feeder 860 capable of feeding and delivering SAP in a substantially continuous layer on top of the central fibrous layer 284. The SAP may be combined with and adhesive 295 or a binder to assist with the placement of the SAP on the central fibrous layer 284. For instance, the free SAP may be mixed with a binder or adhesive prior to combination with the central fibrous layer 284, or a layer of adhesive may be disposed between the upper layer 280 of free SAP and the central fibrous layer 284. The SAP particles may then become affixed to the adhesive 295 (and to the central fibrous layer 284) when the absorbent core 28 is passed through the one or more nip rollers 821 at the core forming station 820. After the upper SAP layer and central fibrous layer 284 are combined, the SAP may migrate into the fibers of the central fibrous layer 284, but preferably, at least some of the SAP remains in the upper layer 280 on top of the central fibrous layer 284.

The absorbent cores 28 are then cut to length by cutting knife 830. Cutting knife 830 can be any suitable cutting device capable of cutting absorbent core 28 of the invention. For example, cutting knife 830 can be comprise of a set of rollers; one being an anvil, and another having a knife attached at one point on the roller whereby the diameter of the roller is selected to coordinate with the speed at which absorbent cores 28 are formed. The knife roller and anvil roller then can rotate at the same speed as the line speed to cut the absorbent core 28 at select areas to form uniform length cores 28. Optionally, the knife roller apparatus may be equipped to crimp or seal the ends of the absorbent cores 28 during the cutting process. Persons of ordinary skill in the art are capable of designing a suitable cutting knife 830 given the specifics of each article forming assembly line.

The absorbent cores 28 then are transported to forming station 800 via core conveyor 880. Top sheet material 24 may be supplied to forming station 800 by top sheet supply mechanism 240, which can be any supply mechanism capable of supplying top sheet 24 to forming station 800. Preferably, top sheet material 24 is supplied via a supply roller and select feed or guide rollers. Back sheet material 26 likewise can be supplied to forming station 800 by back sheet supply mechanism 260, which can be any supply mechanism capable of supplying back sheet 26 to forming station 800. Preferably, back sheet material 26 is supplied via a supply roller and select feed or guide rollers. Forming station brings together the respective components of absorbent article 10 by disposing absorbent laminate core 28 between top sheet material 24, and back sheet material 26. The final absorbent article 10 may then be cut and folded to the appropriate size and shape downstream from forming station 800, as indicated by the right-facing arrow in FIGS. 6 and 7.

Other embodiments, uses, and advantages of the invention will be apparent to those skilled in the art from consideration of the specification and practice of the invention disclosed herein. The specification should be considered exemplary only, and the scope of the invention is accordingly intended to be limited only by the following claims.

What is claimed is:

1. An absorbent core for a disposable absorbent article comprising:
a central fibrous layer having synthetic fibers;
an upper layer disposed on top of the central fibrous layer comprising a first superabsorbent polymer in free form; and
a lower layer disposed beneath the central fibrous layer comprising a substantially continuous layer of a second superabsorbent polymer;
wherein the first superabsorbent polymer has different superabsorbent properties than the second superabsorbent polymer.

2. The absorbent core of claim 1, where the synthetic fibers are tow fibers.

3. The absorbent core of claim 1, where the synthetic fibers are selected from the group consisting of cellulose ester fibers, cellulose acetate fibers, rayon fibers, lyocell fibers, polyacrylonitrile fibers, polyester fibers, polypropylene fibers, polyethylene fibers, and mixtures and combinations thereof.

4. The absorbent core of claim 3, where the synthetic fibers are cellulose ester fibers.

5. The absorbent core of claim 3, where the synthetic fibers are cellulose acetate fibers.

6. The absorbent core of claim 3, where the synthetic fibers are polypropylene fibers.

7. The absorbent core of claim 1, where the synthetic fibers are substantially continuous fibers.

8. The absorbent core of claim 7, where the length of the synthetic fibers is substantially equal to the length of the absorbent core.

9. The absorbent core of claim 1, where the synthetic fibers are discontinuous fibers.

10. The absorbent core of claim 9, where the synthetic fibers are formed into a carded non-woven web.

11. The absorbent core of claim 1, where the lower layer further comprises a substrate layer, on which is disposed the substantially continuous layer of superabsorbent polymer.

12. The absorbent core of claim 11, where the substrate layer is a non-woven web.

13. The absorbent core of claim 11, where the substrate layer is an airlaid web.

14. The absorbent core of claim 11, where the substrate layer is a tissue.

15. The absorbent core of claim 1, wherein the absorbent core further comprises at least one containment layer.

16. The absorbent core of claim 15, where the at least one containment-layer comprises a material selected from the group consisting of: a tissue, a nonwoven, a polyolefin film and combinations and fragments thereof.

17. The absorbent core of claim 15, where the absorbent core comprises two containment layers, where one containment layer is disposed on top of the absorbent core, and the other containment layer is disposed underneath the absorbent core.

18. The absorbent core of claim 1, wherein the absorbent core further comprises at least one additional layer.

19. The absorbent core of claim 18, where the at least one additional layer is selected from the group consisting of: a fluid transfer layer, a fluid handling layer, a storage layer, a wicking layer, a fluid distribution layer, and combinations and fragments thereof.

20. The absorbent core of claim 1, wherein the first superabsorbent polymer is a high-AUL superabsorbent polymer.

21. The absorbent core of claim 1, wherein the second superabsorbent polymer is a fast-absorbing superabsorbent polymer.

22. The absorbent core of claim 1,. wherein the first superabsorbent polymer is a high-AUL superabsorbent polymer, and the second superabsorbent polymer is a fast-absorbing superabsorbent polymer.

23. The absorbent core of claim 1, wherein the upper layer comprises a virgin superabsorbent polymer.

24. The absorbent core of claim 1, wherein the upper layer comprises a blend of two or more types of superabsorbent polymer.

25. The absorbent core of claim 24, wherein the upper layer comprises a blend of a high-AUL superabsorbent polymer and a permeable superabsorbent polymer.

26. The absorbent core of claim 24, wherein the upper layer comprises a blend of a fast absorbing superabsorbent polymer and a permeable superabsorbent polymer.

27. The absorbent core of claim 1, wherein the lower layer comprises a virgin superabsorbent polymer.

28. The absorbent core of claim 1, wherein the lower layer comprises a blend of two or more types of superabsorbent polymer.

29. The absorbent core of claim 28, wherein the lower layer comprises a blend of a fast absorbing superabsorbent polymer and a permeable superabsorbent polymer.

30. The absorbent core of claim 28, wherein the lower layer comprises a blend of a fast absorbing superabsorbent polymer and a high-AUL superabsorbent polymer.

31. The absorbent core of claim 29, wherein the upper layer comprises a blend of a high-AUL superabsorbent polymer and a permeable superabsorbent polymer.

32. An absorbent core for a disposable absorbent article comprising:
a central fibrous layer having synthetic fibers; and
a lower layer disposed beneath the central fibrous layer comprising a substantially continuous layer of a superabsorbent polymer;
wherein the lower layer comprises a blend of a first superabsorbent polymer and a second superabsorbent polymer;
wherein the first superabsorbent polymer has different absorbent properties than the second superabsorbent polymer.

33. The absorbent core of claim 32, where the synthetic fibers are tow fibers.

34. The absorbent core of claim 32, where the synthetic fibers are selected from the group consisting of cellulose ester fibers, cellulose acetate fibers, rayon fibers, lyocell fibers, polyacrylonitrile fibers, polyester fibers, polypropylene fibers, polyethylene fibers, and mixtures and combinations thereof.

35. The absorbent core of claim 34, where the synthetic fibers are cellulose ester fibers.

36. The absorbent core of claim 34, where the synthetic fibers are cellulose acetate fibers.

37. The absorbent core of claim 34, where the synthetic fibers are polypropylene fibers.

38. The absorbent core of claim 32, where the synthetic fibers are substantially continuous fibers.

39. The absorbent core of claim 38, where the length of the synthetic fiber is substantially equal to the length of the absorbent core.

40. The absorbent core of claim 32, where the synthetic fibers are discontinuous fibers.

41. The absorbent core of claim 40, where the synthetic fibers are formed into a carded non-woven web.

42. The absorbent core of claim 32, where the lower layer further comprises a substrate layer, on which is disposed the substantially continuous layer of superabsorbent polymer.

43. The absorbent core of claim 42, where substrate layer is a non-woven web.

44. The absorbent core of claim 42, where substrate layer is an airlaid web.

45. The absorbent core of claim 42, where substrate layer is a tissue.

46. The absorbent core of claim 32, wherein the absorbent core further comprises at least one containment layer.

47. The absorbent core of claim 46, where the at least one containment layer comprises a material selected from the group consisting of: a tissue, a nonwoven, a polyolefin film, and combinations and fragments thereof.

48. The absorbent core of claim 32, wherein the absorbent core further comprises at least one additional layer.

49. The absorbent core of claim 48, where the additional layer is selected from the group consisting of: a fluid transfer layer, a fluid handling layer, a storage layer, a wicking layer, a fluid distribution layer, and combinations and fragments thereof.

50. The absorbent core of claim 32, wherein the first superabsorbent polymer is a fast absorbing superabsorbent polymer.

51. The absorbent core of claim 32, wherein the second superabsorbent polymer is a permeable superabsorbent polymer.

52. The absorbent core of claim 32, wherein the first superabsorbent polymer is a fast-absorbing superabsorbent polymer, and the second superabsorbent polymer is a permeable superabsorbent polymer.

53. The absorbent core of claim 32, wherein the first superabsorbent polymer is a fast-absorbing superabsorbent polymer, and the second superabsorbent polymer is a high-AUL superabsorbent polymer.

54. An absorbent core for a disposable absorbent article comprising:
a central fibrous layer having synthetic fibers; and
an upper layer disposed on top of the central fibrous layer comprising a substantially continuous layer of superabsorbent polymer;
wherein the upper layer comprises a blend of a first superabsorbent polymer and a second superabsorbent polymer;
wherein the first superabsorbent polymer has different absorbent properties than the second superabsorbent polymer.

55. The absorbent core of claim 54, where the synthetic fibers are tow fibers.

56. The absorbent core of claim 54, where the synthetic fibers are selected from the group consisting of cellulose ester fibers, cellulose acetate fibers, rayon fibers, lyocell fibers, polyacrylonitrile fibers, polyester fibers, polypropylene fibers, polyethylene fibers, and mixtures and combinations thereof.

57. The absorbent core of claim 56, where the synthetic fibers are cellulose ester fibers.

58. The absorbent core of claim 56, where the synthetic fibers are cellulose acetate fibers.

59. The absorbent core of claim 56, where the synthetic fibers are polypropylene fibers.

60. The absorbent core of claim 54, where the synthetic fibers are substantially continuous fibers.

61. The absorbent core of claim 60, where the length of the synthetic fiber is substantially equal to the length of the absorbent core.

62. The absorbent core of claim 54, where the synthetic fibers are discontinuous fibers.

63. The absorbent core of claim 54, where the lower layer further comprises a lower tissue layer, on which is disposed the substantially continuous layer of superabsorbent polymer.

64. The absorbent core of claim 54, where the lower layer further comprises a lower non-woven layer, on which is disposed the substantially continuous layer of superabsorbent polymer.

65. The absorbent core of claim 54, where the lower layer further comprises an airlaid layer, on which is disposed the substantially continuous layer of superabsorbent polymer.

66. The absorbent core of claim 54, wherein the absorbent core further comprises at least one containment layer.

67. The absorbent core of claim 66, where the at least one containment layer comprises a material selected from the group consisting of: a tissue, a nonwoven, a polyolefin film, and combinations and fragments thereof.

68. The absorbent core of claim 54, wherein the absorbent core further comprises at least one additional layer.

69. The absorbent core of claim 68, where the additional layer is selected from the group consisting of: a fluid transfer layer, a-fluid handling layer, a storage layer, a wicking layer, a fluid distribution layer, and combinations and fragments thereof.

70. The absorbent core of claim 54, wherein the lower first superabsorbent polymer is a high-AUL superabsorbent polymer.

71. The absorbent core of claim 54, wherein the second superabsorbent polymer is a permeable superabsorbent polymer.

72. The absorbent core of claim 54, wherein the first superabsorbent polymer is a high-AUL superabsorbent polymer, and the second superabsorbent polymer is a permeable superabsorbent polymer.

73. The absorbent core of claim 54, wherein the first superabsorbent polymer is a fast absorbing superabsorbent polymer, and the second superabsorbent polymer is a permeable superabsorbent polymer.

74. An absorbent article comprising:
a liquid permeable topsheet;
a liquid impermeable backsheet;
an absorbent core disposed between the topsheet and backsheet;
wherein the absorbent core comprises
a central fibrous layer having synthetic fibers;
an upper layer disposed on top of the central fibrous layer comprising a first superabsorbent polymer in free form; and
a lower layer disposed beneath the central fibrous layer comprising a substantially continuous layer of a second superabsorbent polymer;
wherein the first superabsorbent polymer has different absorbent properties than the second superabsorbent polymer.

75. The absorbent article of claim 74, where the synthetic fibers are tow fibers.

76. The absorbent article of claim 74, where the synthetic fibers are selected from the group consisting of cellulose ester fibers, cellulose acetate fibers, rayon fibers, lyocell fibers, polyacrylonitrile fibers, polyester fibers, polypropylene fibers, polyethylene fibers, and mixtures and combinations thereof.

77. The absorbent article of claim 76, where the synthetic fibers are cellulose ester fibers.

78. The absorbent article of claim 76, where the synthetic fibers are cellulose acetate fibers.

79. The absorbent article of claim 76, where the synthetic fibers are polypropylene fibers.

80. The absorbent article of claim 74, where the synthetic fibers are substantially continuous fibers.

81. The absorbent article of claim 80, where the length of the synthetic fibers is substantially equal to the length of the absorbent core.

82. The absorbent article of claim 74, where the synthetic fibers are discontinuous fibers.

83. The absorbent article of claim 82, where the synthetic fibers are formed into a carded non-woven web.

84. The absorbent article of claim 74, where the lower layer further comprises a substrate layer, on which is disposed the substantially continuous layer of superabsorbent polymer.

85. The absorbent article of claim 84, where the substrate layer is a non-woven web.

86. The absorbent article of claim 84, where the substrate layer is an airlaid web.

87. The absorbent article of claim 84, where the substrate layer is a tissue.

88. The absorbent article of claim 74, wherein the absorbent core further comprises at least one containment layer.

89. The absorbent article of claim 88, where the at least one containment layer comprises a material selected from the group consisting of: a tissue, a nonwoven, a polyolefin film and combinations and fragments thereof.

90. The absorbent article of claim 88, where the absorbent core comprises two containment layers, where one containment layer is disposed on top of the absorbent core, and the other containment layer is disposed underneath the absorbent core.

91. The absorbent article of claim 74, wherein the absorbent core further comprises at least one additional layer.

92. The absorbent article of claim 91, where the at least one additional layer is selected from the group consisting of: a fluid transfer layer, a fluid handling layer, a storage layer, a wicking layer, a fluid distribution layer, and combinations and fragments thereof.

93. The absorbent article of claim 74, wherein the first superabsorbent polymer is a high-AUL superabsorbent polymer.

94. The absorbent article of claim 74, wherein the second superabsorbent polymer is a fast-absorbing superabsorbent polymer.

95. The absorbent article of claim 74, wherein the first superabsorbent polymer is a high-AUL superabsorbent polymer, and the second superabsorbent polymer is a fast-absorbing superabsorbent polymer.

96. The absorbent article of claim 74, wherein the upper layer comprises a virgin superabsorbent polymer.

97. The absorbent article of claim 74, wherein the upper layer comprises a blend of two or more types of superabsorbent polymer.

98. The absorbent article of claim 97, wherein the upper layer comprises a blend of a high-AUL superabsorbent polymer and a permeable superabsorbent polymer.

99. The absorbent article of claim 97, wherein the upper layer comprises a blend of a fast absorbing superabsorbent polymer and a permeable superabsorbent polymer.

100. The absorbent article of claim 74, wherein the lower layer comprises a virgin superabsorbent polymer.

101. The absorbent article of claim 74, wherein the lower layer comprises a blend of two or more types of superabsorbent polymer.

102. The absorbent article of claim 101, wherein the lower layer comprises a blend of a fast absorbing superabsorbent polymer and a permeable superabsorbent polymer.

103. The absorbent article of claim 101, wherein the lower layer comprises a blend of a fast absorbing superabsorbent polymer and a high-AUL superabsorbent polymer.

104. The absorbent article of claim 103, wherein the upper layer comprises a blend of a high-AUL superabsorbent polymer and a permeable superabsorbent polymer.

105. The absorbent article of claim 74, whereby the article has a first waist region, a second waist region longitudinally opposed to the first waist region, and a crotch region between the first and second waist regions, the article further comprising at least one fastening element attached to a lateral edge of the first waist region; and
one or more target devices attached to the article in the second waist region, where at least one fastening element and the one or more target devices are capable of attaching to one another, the one or more target devices being located so that the first waist region and second waist region of the garment may be joined to one another to secure the garment on the wearer.

106. The absorbent article of claim 105, further comprising elastic leg gathers comprising one or more elastic materials disposed adjacent a lateral edge of the crotch region, and standing leg gathers disposed on the top sheet adjacent the lateral edge of the crotch region.

107. The absorbent core of claim 105, wherein the at least one fastening element comprises a hook portion of a hook and loop fastener and the one or more target devices comprise the loop portion of a hook and loop fastener.

108. The absorbent article of claim 105, wherein the at least one fastening element is an adhesive tape and the one or more target devices comprise a tape receiving surface.

109. The absorbent article of claim 105, wherein the at least one fastening element is comprised of a pair of laterally extending tabs disposed on the lateral edges of the first waist region, whereby the laterally extending tabs each include at least one fastening element.

110. The absorbent article of claim 74, further comprising at least one additional layer.

111. The absorbent article of claim 110, wherein the at least one additional layer is selected from the group consisting of: a fluid transfer layer, a fluid handling layer, a storage layer, a wicking layer, a fluid distribution layer, and combinations and fragments thereof.

112. The absorbent article of claim 110, wherein at least one additional layer is disposed between the absorbent core and the top sheet.

113. The absorbent article of claim 110, wherein the at least one additional layer disposed between the absorbent core and the back sheet.

114. A method of manufacturing a disposable absorbent article comprising the steps of:
a) providing a liquid impermeable back sheet and a liquid permeable top sheet;
b) preparing an absorbent core by:
b1) providing a central fibrous layer having synthetic fibers;
b2) providing an upper layer comprised primarily of a first superabsorbent polymer;
b3) providing a lower layer comprised of a substantially continuous layer of a second superabsorbent polymer having different absorbent properties than the first superabsorbent polymer; and
b4) disposing the central fibrous layer at least partially between the upper layer and the lower layer; and
c) disposing the absorbent core at least partially between the top sheet and the back sheet.

115. The method of claim 114, where the synthetic fibers are tow fibers.

116. The method of claim 114, whereby providing the upper layer comprises providing the first superabsorbent polymer in free form to the absorbent core.

117. The method of claim 114, further comprising disposing at least one additional layer between the absorbent laminate core and the top sheet.

118. The method of claim 117, where the additional layer is selected from the group consisting of: a fluid transfer layer, a fluid handling layer, a storage layer, a wicking layer, a fluid distribution layer, and combinations and fragments thereof.

119. The method of claim 114, further comprising disposing at least one additional layer between the absorbent laminate core and the back sheet.

120. The method of claim 119, where the additional layer is selected from the group consisting of: a fluid transfer layer, a fluid handling layer, a storage layer, a wicking layer, a fluid distribution layer, and combinations and fragments thereof.

121. The method of claim 114, wherein providing a central fibrous layer comprises providing continuous synthetic fiber, and opening the synthetic fiber to form a fibrous matrix.

122. The method of claim 114, wherein providing a central fibrous layer comprises providing a carded web of synthetic fiber.

123. The method of claim 114, wherein providing the central fibrous layer comprises providing synthetic fibers selected from the group consisting of cellulose ester fibers, cellulose acetate fibers, rayon fibers, lyocell fibers, polyacrylonitrile fibers, polyester fibers, polypropylene fibers, polyethylene fibers, and mixtures and combinations thereof.

124. The method of claim 123, wherein the synthetic fibers are cellulose ester fibers.

125. The method of claim 114, further comprising disposing at least one containment layer between the absorbent core and either the top sheet or the back sheet.

126. The method of claim 125, wherein the at least one containment layer comprises a material selected from the group consisting of: a tissue, a nonwoven, a polyolefin film and combinations and fragments thereof.
